(19) United States
(12) Patent Application Publication
(10) Pub. No.: US 2011/0184314 A1
(43) Pub. Date: Jul. 28, 2011

(54) ENDOCERVICAL LAVAGE DEVICE AND METHOD

(76) Inventor: Mario S. Gonzalez, Princeton, NJ (US)

(21) Appl. No.: 13/012,542

(22) Filed: Jan. 24, 2011

Related U.S. Application Data
(60) Provisional application No. 61/298,139, filed on Jan. 25, 2010.
(60) Provisional application No. 61/298,136, filed on Jan. 25, 2010.

(51) Int. Cl. A61B 10/02

(52) U.S. Cl. 600/569

(57) ABSTRACT

An endocervical lavage device for retrieving cervical and endocervical cells for cytologic examination and to method(s) for retrieving cervical and endocervical cells and for using the device. The device generally comprises a multi-lumen tube having a proximal end and a distal end. The distal end of the multi-lumen tube is to be introduced into the uterine cavity of a uterus of a female. An inflatable structure is mounted at the distal end of the multi-lumen tube for creating a first seal between the uterine cavity and an internal cervical os of a cervical canal, the cervical canal being defined at its ends by the internal os at an endocervix that leads into the uterine cavity and an external os at an ectocervix. The device further comprises a collecting structure for advancement distally along the tube to engage the ectocervix, to create a second seal about the external cervical os.

### CROSS-REFERENCE TO RELATED APPLICATIONS

This application claims the benefit of U.S. provisional patent application Ser. No. 61/298,136, filed Jan. 25, 2010, and U.S. provisional patent application Ser. No. 61/298,139, filed Jan. 25, 2010, each of which is incorporated herein by reference in its entirety.

### FIELD OF THE INVENTION

The present invention relates to a device and to a method for sampling and collecting a woman's endocervical cells.

### BACKGROUND OF THE INVENTION

In the U.S. alone, invasive cancer of the cervix is a condition that afflicts more than 13,000 women a year causing 4000 deaths annually. Pre-invasive carcinoma or carcinoma in situ affects another 50,000 women every year. One reason for the relatively high mortality is because cervical cancer can be asymptomatic and that the endocervical canal is difficult to sample and thus detecting abnormalities via testing can be a critical tool to reduce mortality, to provide for earlier diagnosis to facilitate management and treatment. There are also between 250,000 and 1 million American women diagnosed with cervical intraepithelial neoplasia (“CIN”), also known as dysplasia, every year. Cervical intraepithelial neoplasia cases may spontaneously regress but many progress to cancer of the cervix.

A Papanicolaou test (“Pap test”) is one method of cervical screening for detecting atypical and dysplastic cells that may be the precursor of cancer or cancer itself. In the US the wide spread use of cervical screening has reduced the incidence of invasive cervical cancer by 50%. However, clinical literature indicates that current methods for collecting cervical and endocervical cells may yield suboptimal samples, particularly those involving or arising within the endocervical canal.

Accordingly, it is estimated that 20% of cervical cancers go undetected at earlier stages of the disease as a result of sample collection deficiencies. These undetected cancers may arise from endocervical and transition zone areas. The transition zone or transformation zone is the specific area of the cervix where most precancerous and cancerous changes occur. This represents the junction between the squamous and glandular epithelium (squamous-columnar junction) or between the ectocervix and the endocervix and endocervical canal.

The surface epithelial cells from the endocervix, particularly those from high within the endocervical canal and submucosal endocervical glands, are typically not accessible by traditional tools such as spatulas and brushes. Further, these areas are not easily visible by colposcopic examination of the cervix.

It would be desirable to provide a device and method that offers a greater opportunity for obtaining cells from the upper endocervical canal and endocervical glands to obtain samples for more accurate and earlier detection, evaluation and assessment of disease progression and extent, management and treatment of residual dysplasia and cervical cancer. Such a device and method could reduce cost of care and need for additional tests and procedures including more costly and invasive surgical procedures, like colposcopy or other surgical procedures such as cervical or cone biopsies and assist in dysplasia and cancer management and monitoring and assessment of disease progression. It would thus be desirable to increase the efficiency and effectiveness during sampling for cytologic screening and greater accuracy and yield in detecting residual dysplasia, cervical cancer and other conditions as compared to conventional methods, thereby facilitating earlier detection and treatment of cervical cancer, and reduction of morbidity and mortality from cervical cancer.

### SUMMARY OF THE INVENTION

The present invention relates to an endocervical lavage device or apparatus for retrieving cervical and endocervical cells for cytologic examination and to a method(s) for retrieving cervical and endocervical cells and for using the endocervical lavage device or apparatus. The device or apparatus generally comprises a multi-lumen tube for introduction into a uterine cavity of a uterus of a female. The multi-lumen tube has a distal end and a proximal end. The distal end of the multi-lumen tube is to be introduced into the uterine cavity. The apparatus further comprises an inflatable structure mounted at the distal end of the multi-lumen tube, for creating a first seal between the uterine cavity and an internal cervical os of a cervical canal, the cervical canal being defined at its ends by the internal os at an endocervix that leads into the uterine cavity and an external os at an ectocervix, and a collecting structure for advancement distally along the tube to engage the ectocervix, to create a second seal about the external cervical os.

In yet another aspect of the present invention, the apparatus comprises: a multi-lumen tube such as a dual lumen tube having a distal end and a proximal end, the dual lumen tube defining channels for a lavage solution such as saline and an inflation fluid such as air; a balloon mounted to the distal end of the dual lumen tube, the balloon having an interior in communication with an air lumen for inflation and deflation of the balloon; a tube channel for the lavage solution having at least one aperture or opening located proximally of the balloon; a carrier tube to which the dual lumen tube is slidably mounted; a collecting structure having a distal opening mounted to the distal end of the carrier tube, the distal opening of the collecting structure being adapted to engage and seal against an ectocervix; and a collector tube extending from the proximal end of the carrier tube and into the interior of the collecting structure to enable liquid and cellular material within the collecting structure to be aspirated.

The present invention also provides a method(s) of retrieving cervical and endocervical cells for cytologic examination. The method(s) generally comprises mechanically pressure washing a sealed cervical canal with a lavage solution to create turbulence thereby dislodging cervical and endocervical cells from surface epithelium or from submucosal endocervical glands, and collecting the cervical and endocervical cells for cytologic examination.

A method of retrieving cervical and endocervical cells for cytologic examination in accordance with the present invention comprises introducing a multi-lumen tube into a uterus comprising a uterine cavity, the multi-lumen tube having a distal end and a proximal end with the distal end of the multi-lumen tube disposed in the uterine cavity; and inflating an inflatable structure at the distal end of the multi-lumen tube, thereby creating a first seal between the uterine cavity and an internal cervical os of a cervical canal, the cervical canal being defined at its ends by the internal os at an endocervix that leads into the uterine cavity and an external os at an ectocervix; advancing a collecting structure distally along said tube to engage the ectocervix, thereby creating a second seal about the external cervical os; introducing a lavage solution through the multi-lumen tube under sufficient pressure and directed so as to create force to dislodge cervical and endocervical cells; collecting the dislodged cells along with the lavage solution in the collecting structure; and retrieving the collected cells and solution from the collecting structure.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, which are not necessarily to scale, wherein:

FIG. 1 is a diagrammatic illustration, partly in section, of the human female reproductive anatomy;

FIG. 2 is an illustration of a device that may be used in the practice of the invention;

FIG. 2A is a sectional illustration seen along the line 2A-2A of FIG. 2.

FIG. 3 is a diagrammatic illustration of the device of FIG. 2 advanced into engagement with the uterus and endocervical cavity in readiness to perform a collection procedure;

FIG. 4 is an enlarged illustration of the uterus and the distal end of the device in the position shown in FIG. 3; and

FIG. 4A is a sectional illustration along the line 4A-4A of FIG. 4.

### DETAILED DESCRIPTION OF THE INVENTION

The following detailed description of the embodiment(s) is merely exemplary in nature and is in no way intended to limit the invention, its application, or uses.

In accordance with the present invention, a lavage solution, such as saline, is directed into the endocervical canal under sufficient force and flow rates to flush out and to dislodge cells from the endocervical canal, preferably along the full length of the canal and the submucosal endocervical glands within the submucosal endocervical stroma. The forces and direction of flow are controlled to dislodge the cells with minimal, if any, damage to preserve their cytologic examination. The cells, together with the saline solution, are collected in a receptacle disposed at the ectocervix. The saline and cells may be withdrawn from the collector by aspiration through a tube. During the procedure, the endocervical canal is sealed at its proximal and distal ends by a collecting structure and an inflatable structure such as a balloon respectively, wherein the balloon is inflated within the uterus adjacent the upper end of the endocervix.

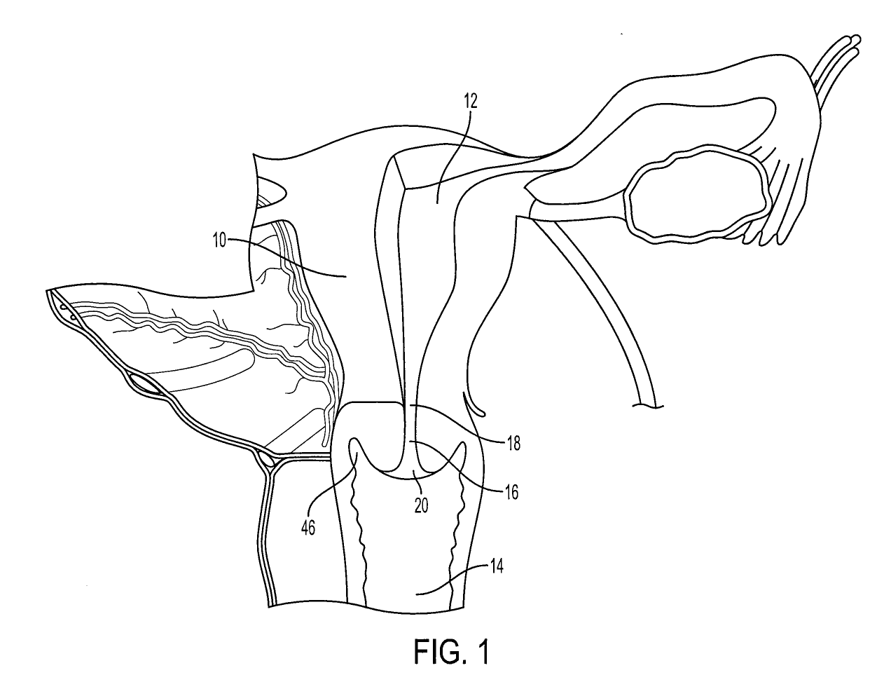

As shown in FIG. 1, the female reproductive anatomy includes the uterus 10 including the uterine cavity 12. The vagina 14 communicates with the uterus 12 through a narrow passage, the cervical canal 16. The cervical canal is defined at its ends by an internal os 18 at the endocervix that leads into the uterine cavity 12 and an external os 20 at the ectocervix that communicates with the vaginal passage 14. An objective of the present invention is to facilitate harvesting of cells from within the cervical canal including endocervical surface epithelium and submucosal endocervical glands 16.

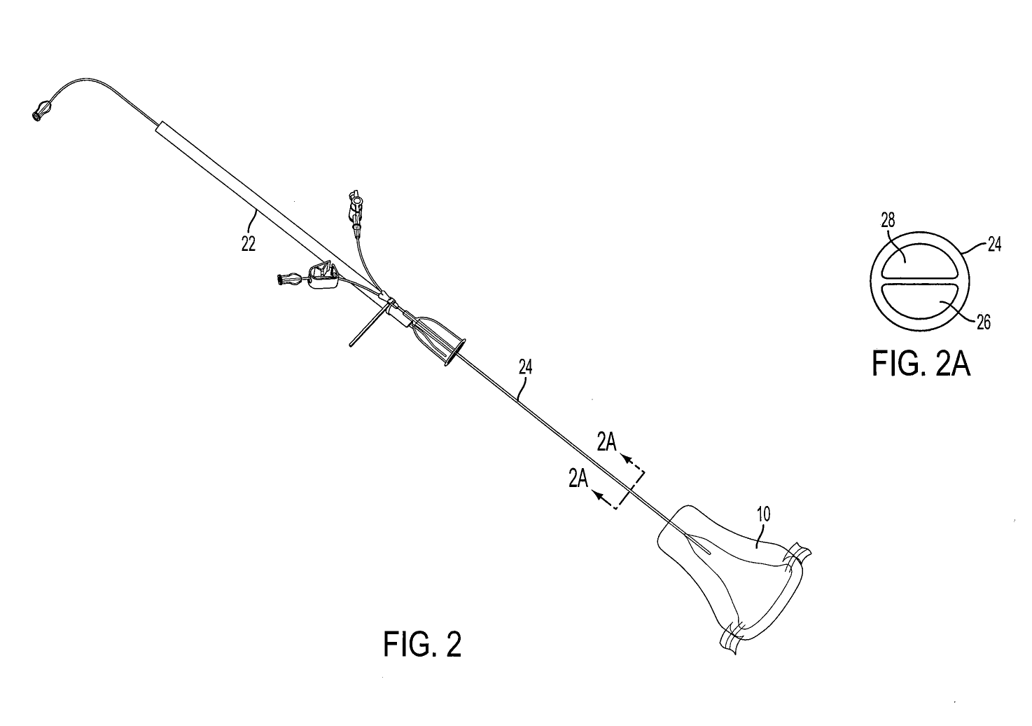

FIGS. 2-4 illustrate a device or apparatus that may be employed in practice of the invention. The device is inserted through the vaginal passage with part of it being advanced through the cervical canal 16 and into the uterus 10 beyond the internal os 18. The device includes a carrier tube 22 that carries the other components of the device. A dual lumen tube 24 is carried by the carrier tube 22 and is mounted for longitudinal movement with respect to the carrier tube 22. For example, a dual lumen tube 24 may be affixed to the carrier tube 22 in such a way that they are both included within a sleeve. The dual lumen tube 24 has two lumens (FIG. 2A) 26 and 28 adapted to carry, respectively, an inflation fluid such as air and a lavage solution such as saline. The proximal end of the dual lumen tube 24 includes a bifurcation junction 30 connected to a pair of tubular tails 32, 34, having fittings 36, 38 to communicate air and saline, respectively. A clamping device, illustrated as a tie wrap 40, is provided to enable the dual lumen tubing 24 to be securely attached to the carrier tube 22 after the device has been positioned, as described below. A collecting structure shown as collector 42, such as a cup-shape device is attached to the distal end of the carrier tube. The collector can be plastic injection molded. The collector 42 preferably includes a guide 45 that opens into the interior of the collector. The guide 45 receives the dual lumen tube to permit the tube 24 to slide through the guide 45.

The distal end of the dual lumen tube includes an inflatable structure such as an inflatable balloon 44, the interior of the balloon being in communication with the air inflation lumen 26. As will be described, when the dual lumen tube 24 has been advanced to the uterine cavity, the balloon is inflated, as by a syringe, connected to the fitting 36. The balloon is inflated sufficiently and is positioned adjacent the internal os 18 to provide a seal between the uterine cavity 12 and the cervical canal 16. In an embodiment, the balloon is comprised of silicone.

The silicone balloon prevents a lavage solution such as saline from escaping into the uterine cavity and creates the upper seal and the collecting cup completes the seal below and serves as a collecting cup for the saline used in the washing that contains the dislodged endocervical cells from the desired areas. With the balloon inflated and positioned, the fitting 36 may be closed, as by a stopcock, to maintain the balloon in an inflated position, sealing the internal end of the cervical canal.

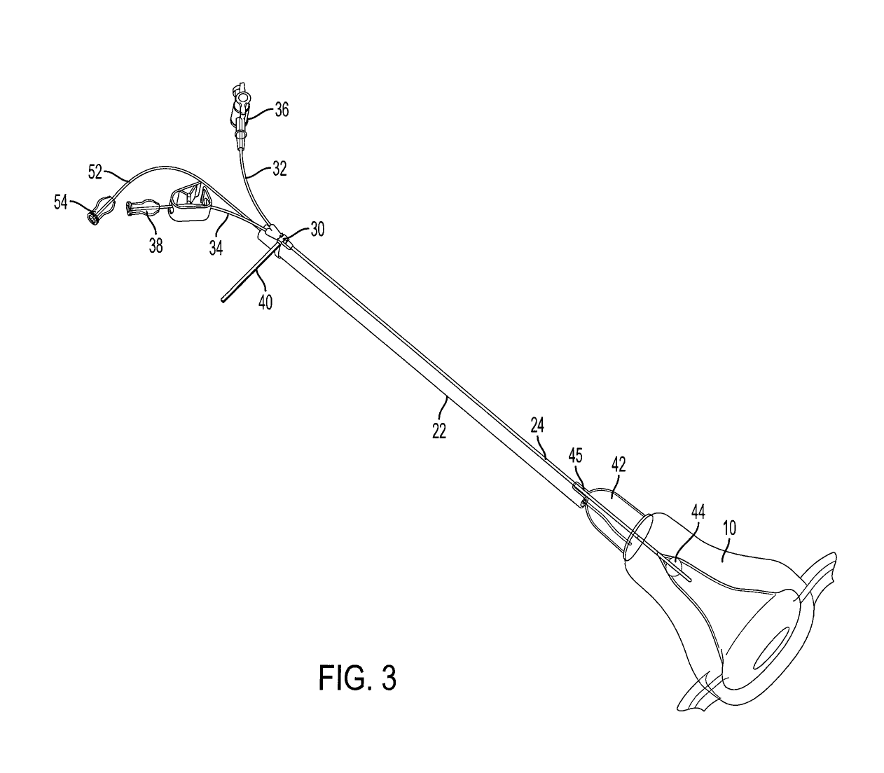

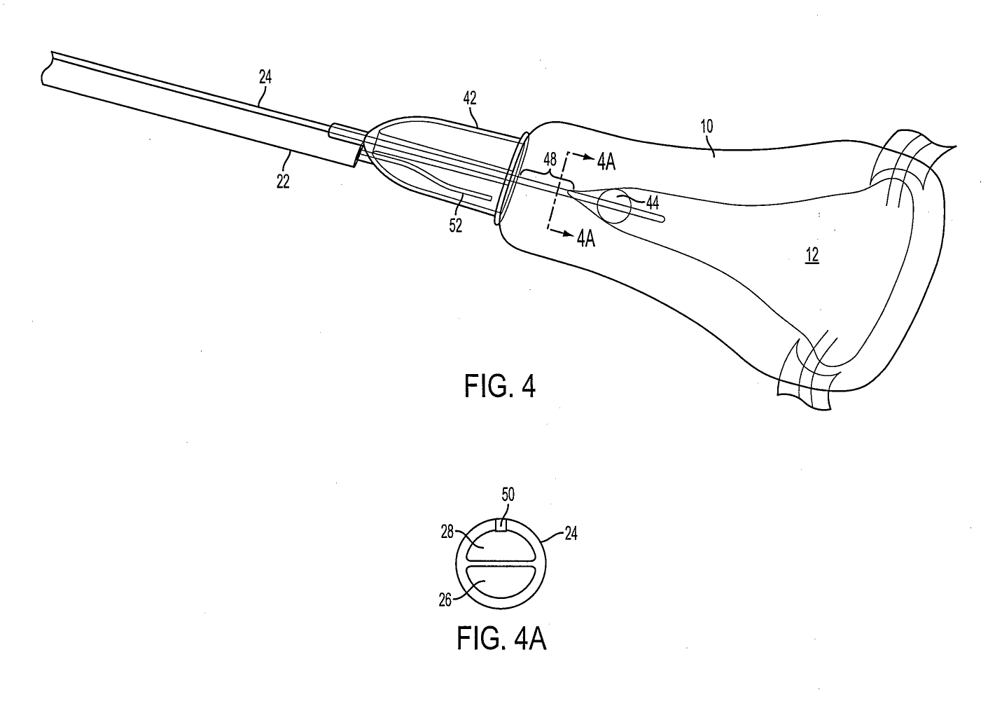

With the balloon inflated, the carrier tube 22 then is advanced, being guided by the cooperation of the guide 45 with the dual lumen tubing 24, until the distal, open end of the cup-like collector 42 is in firm sealing engagement with the ectocervix (lateral formix) 46. The collector 42 is pushed into firm sealing engagement with the lateral formix, thus drawing the balloon and collector toward each other sealing the cervical canal internally and externally. As shown in FIGS. 3 and 4, an intermediate portion 48 of the dual lumen tube 24 proximal of the balloon is adapted to extend through the cervical canal 16 when the device is secured and sealed in place. This intermediate segment 48 (FIG. 4) is provided with one or more openings (FIG. 4A) that communicate with the saline lumen 28 and enable saline to be injected through the saline lumen 28 and out through the aperture or apertures 50 (FIG. 4A). The aperture(s) may be variably designed provided that it enables saline to be injected through the aperture(s) under a sufficient pressure, force and direction to maximize the region and amount of cells that are dislodged and may be collected. The retrieved saline solution contains cells from the endocervix, ectocervix, internal os and the external os.

This creates maximum pressure and an irrigation effect on the epithelium to facilitate the dislodging of the cells. The pressure washing of the sealed canal also creates expansion, turbulence and increased mechanical pressure and further dislodging of cells from the surface epithelium. The mechanical effect caused by the saline helps distend and dilate and open the submucosal endocervical glands and allow for fluid to enter these microscopic spaces. Here, the turbulence and increased mechanical pressure within these glands help also to dislodge endocervical cells from these glands.

The invention embodies a “pressured washing” application that targets and samples the endocervical canal. The device seals the canal and creates an ideal isolated environment for accessing, dislodging and collecting the cells from otherwise inaccessible or difficult to sample areas: the endocervical surface epithelial cells within the entire length of the endocervical canal and the submucosal endocervical glands within the submucosal endocervical stroma.

The harvested material is collected through a collection tube 52 that may extend through the carrier tube 22. The distal end of the collection tube terminates in one or more openings within the collector 42. The proximal end of the collector tube 52 extends out of the proximal end of the carrier tube 22 and is provided with a fitting 54 to which an aspiration syringe (not shown) may be attached. The collected material thus may be withdrawn from the collector to enable the cells and other matter of interest to be examined. At the conclusion of the procedure, the balloon 44 may be deflated and the entire apparatus may be withdrawn.

The invention described herein discloses an important device and method for the collection of cervical and endocervical cells for cytologic examination by Thin-prep or conventional cytology and Pap test for the analysis, diagnosis, management and surveillance of squamous dysplasia involving the high endocervical canal, assessment and management of residual dysplasia, diagnosis of endocervical precancerous lesions, and importantly to improve the collection and yield of endocervical cells within the endocervical canal and endocervical glands deep from endocervical stroma and improve the efficacy of the diagnosis of glandular neoplasia of the cervix.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A method of retrieving cervical and endocervical cells for cytologic examination, the method comprising:
introducing a multi-lumen tube into a uterus comprising a uterine cavity, the multi-lumen tube having a distal end and a proximal end, the distal end of the multi-lumen tube disposed in the uterine cavity;
inflating an inflatable structure at the distal end of the multi-lumen tube, thereby creating a first seal between the uterine cavity and an internal cervical os of a cervical canal, the cervical canal being defined at its ends by the internal os at an endocervix that leads into the uterine cavity and an external os at an ectocervix;
advancing a collecting structure distally along said tube to engage the ectocervix, thereby creating a second seal about the external cervical os;
introducing a lavage solution through the multi-lumen tube under sufficient pressure and directed so as to create force to dislodge cervical and endocervical cells;
collecting the dislodged cells along with the lavage solution in the collecting structure; and
retrieving the collected cells and solution from the collecting structure.

2. The method of claim 1, wherein the lavage solution is a saline solution.

3. The method of claim 1, wherein the multi-lumen tube includes a carrier tube.

4. The method of claim 3, wherein the multi-lumen tube is mounted for longitudinal movement with respect to the carrier tube.

5. The method of claim 1, wherein the multi-lumen tube is a dual lumen tube.

6. The method of claim 5, wherein the dual lumen tube comprises two lumens adapted to carry an inflation fluid and the lavage solution.

7. The method of claim 6, wherein the proximal end of the dual lumen tube includes a bifurcation junction connected to a pair of tubular tails having fittings to communicate air and the lavage solution.

8. The method of claim 3, further comprising providing a clamping device to enable the multi-lumen tube to be securely attached to the carrier tube after the clamping device has been positioned.

9. The method of claim 1, wherein the collecting structure is a cup-shaped device.

10. The method of claim 1, wherein the collecting structure is formed of injection molded plastic.

11. The method of claim 3, wherein the collecting structure is attached to a distal end of the carrier tube.

12. The method of claim 1, wherein the collecting structure includes a guide that opens into an interior of the collecting structure.

13. The method of claim 12, wherein the guide receives the multi-lumen tube to permit the tube to slide through the guide.

14. The method of claim 1, wherein the inflatable structure is a balloon.

15. The method of claim 14, wherein an interior of the balloon is in communication with an air inflation lumen.

16. The method of claim 14, further comprising inflating the balloon by a syringe to provide a seal between the uterine cavity and the cervical canal.

17. The method of claim 14, wherein the balloon comprises silicone.

18. The method of claim 16, wherein the balloon creates an upper seal.

19. The method of claim 16, wherein the collecting structure creates a lower seal.

20. The method of claim 14, further comprising closing a fitting to maintain the balloon in an inflated position.

21. The method of claim 3, further comprising advancing the carrier tube until the distal, open end of the collecting structure is in a firm sealing engagement with the ectocervix.

22. The method of claim 14, wherein an intermediate portion of the multi-lumen tube proximal of the balloon is adapted to extend through the cervical canal when the multi-lumen tube is secured and sealed in place.

23. The method of claim 22, wherein the intermediate portion is provided with one or more openings that communicate with the lavage solution enabling the lavage solution to be injected through the lavage solution lumen and out through the openings.

24. The method of claim 3, further comprising retrieving said collected cells through a collection tube extending through the carrier tube.

25. The method of claim 24, wherein a distal end of the collection tube terminates in one or more openings within the collecting structure.

26. The method of claim 24, wherein a proximal end of the collection tube extends out of the proximal end of the carrier tube and is provided with a fitting.

27. The method of claim 6, wherein the inflation fluid is air.

28. An apparatus for retrieving cervical and endocervical cells for cytologic examination, the apparatus comprising:
a multi-lumen tube for introduction into a uterus comprising a uterine cavity, the multi-lumen tube having a distal end and a proximal end, the distal end of the multi-lumen tube for introduction in the uterine cavity;
an inflatable structure mounted at the distal end of the multi-lumen tube, for creating a first seal between the uterine cavity and an internal cervical os of a cervical canal, the cervical canal being defined at its ends by the internal os at an endocervix that leads into the uterine cavity and an external os at an ectocervix; and
a collecting structure for advancement distally along the tube to engage the ectocervix, to create a second seal about the external cervical os.

29. The apparatus of claim 28, wherein the multi-lumen tube includes a carrier tube.

30. The apparatus of claim 29, wherein the multi-lumen tube is mounted for longitudinal movement with respect to the carrier tube.

31. The apparatus of claim 28, wherein the multi-lumen tube is a dual lumen tube.

32. The apparatus of claim 32, wherein the dual lumen tube comprises two lumens adapted to carry an inflation fluid and a lavage solution.

33. The apparatus of claim 32, wherein the lavage solution is a saline solution.

34. The apparatus of claim 33, wherein the proximal end of the dual lumen tube includes a bifurcation junction connected to a pair of tubular tails having fittings to communicate air and the lavage solution.

35. The apparatus of claim 30, further comprising a clamping device for securing the multi-lumen tube to the carrier tube.

36. The apparatus of claim 28, wherein the collecting structure is a cup-shaped device.

37. The apparatus of claim 28, wherein the collecting structure is formed of injection molded plastic.

38. The apparatus of claim 30, wherein the collecting structure is attached to a distal end of the carrier tube.

39. The apparatus of claim 28, wherein the collecting structure includes a guide that opens into an interior of the collecting structure.

40. The apparatus of claim 39, wherein the guide receives the multi-lumen tube to permit the tube to slide through the guide.

41. The apparatus of claim 28, wherein the inflatable structure is a balloon.

42. The apparatus of claim 41, wherein an interior of the balloon is in communication with an air inflation lumen.

43. The apparatus of claim 41, further comprising a fitting to maintain the balloon in an inflated position.

44. The apparatus of claim 41, wherein an intermediate portion of the multi-lumen tube proximal of the balloon is adapted to extend through the cervical canal when the multi-lumen tube is secured and sealed in place.

45. The apparatus of claim 32, wherein an intermediate portion of the multi-lumen tube is provided with one or more openings that communicate with the lavage solution enabling the lavage solution to be injected through one of the lumens and out through the openings.

46. The apparatus of claim 28, wherein the lavage solution is injected with a syringe.

47. The apparatus of claim 30, further comprising a collection tube extending through the carrier tube for retrieving the collected cells.

48. The apparatus of claim 47, wherein a distal end of the collection tube terminates in one or more openings within the collecting structure.

49. The apparatus of claim 47, wherein a proximal end of the collection tube extends out of the proximal end of the carrier tube and is provided with a fitting.

50. The apparatus of claim 32, wherein the inflation fluid is air.

51. An apparatus for retrieving cervical and endocervical cells for cytologic examination comprising:
a multi-lumen tube having a distal end and a proximal end, the multi-lumen tube defining channels for a lavage solution and an inflation fluid;
a balloon mounted to the distal end of the multi-lumen tube, the balloon having an interior in communication with an air lumen for inflation and deflation of the balloon;
a tube channel for the lavage solution having at least one aperture or opening located proximally of the balloon;
a carrier tube to which the multi-lumen tube is slidably mounted;
a collecting structure having a distal opening mounted to the distal end of the carrier tube, the distal opening of the collecting structure being adapted to engage and seal against an ectocervix; and
a collector tube extending from the proximal end of the carrier tube and into the interior of the collecting structure to enable liquid and cellular material within the collecting structure to be aspirated.

52. The apparatus of claim 51, further comprising a clamping device for securing the multi-lumen tube in relative longitudinal position with respect to the carrier tube.

53. The apparatus of claim 59, wherein the clamping device is a tie wrap.

54. The apparatus of claim 59, wherein the lavage solution is a saline solution.

55. The apparatus of claim 51, wherein the multi-lumen tube is a dual lumen tube.

56. The apparatus of claim 55, wherein the proximal end of the dual lumen tube includes a bifurcation junction connected to a pair of tubular tails having fittings to communicate an inflation fluid and the lavage solution.

57. The apparatus of claim 51, wherein the collecting structure is a cup-shaped device.

58. The apparatus of claim 51, wherein the collecting structure is formed of injection molded plastic.

59. The apparatus of claim 51, wherein the collecting structure includes a guide that opens into an interior of the collecting structure.

60. The apparatus of claim 59, wherein the guide receives the multi-lumen tube to permit the tube to slide through the guide.

61. The apparatus of claim 51, wherein the balloon comprises silicone.

62. The apparatus of claim 51, wherein an intermediate portion of the multi-lumen tube proximal of the balloon is adapted to extend through a cervical canal when the multi-lumen tube is secured and sealed in place.

63. The apparatus of claim 51, wherein an intermediate portion is provided with one or more openings that communicate with the lavage solution enabling the lavage solution to be injected through the lumen and out through the openings.

64. The apparatus of claim 51, wherein a distal end of the collection tube terminates in one or more openings within the collecting structure.

65. The apparatus of claim 51, wherein the inflation fluid is air.

66. An apparatus for retrieving cervical and endocervical cells for cytologic examination, the apparatus comprising:
a first means for creating a first seal in a uterine cavity of a uterus, the first seal adjacent to an endocervix;
a second means for creating a second seal, the second seal engaging an ectocervix thereby to seal a cervical canal;
a means for introducing a lavage solution into the cervical canal such that the lavage solution forcefully contacts cervical and endocervical tissue, thereby dislodging cervical and endocervical cells into the lavage solution to form a collected sample; and
a means for collecting the collected sample for cytologic examination.

67. A method of retrieving cervical and endocervical cells for cytologic examination, the method comprising:
mechanically pressure washing a sealed cervical canal with a lavage solution to create turbulence thereby dislodging cervical and endocervical cells from surface epithelium or from submucosal endocervical glands, and
collecting the cervical and endocervical cells for cytologic examination.
